(19) United States
(12) Patent Application Publication
(10) Pub. No.: US 2003/0199576 A1
(43) Pub. Date: Oct. 23, 2003

(54) Stabilized pure vitamin C in powder-to-liquid form using multi-encapsulation method

(76) Inventors: Chang Eon Lee, Walnut, CA (US); In Sung Yang, Burbank, CA (US)

Correspondence Address: IN SUNG YANG, 628 SOUTH PALM AVENUE, ALHAMBRA, CA, 91803, US

(21) Appl. No.: 10/122,360

(22) Filed: Apr. 15, 2002

(51) Int. Cl. A61K 31/375; A61K 9/127

(52) U.S. Cl. 514/474; 424/450

(57) ABSTRACT

The invention relates to cosmetic powder that contains stabilized Pure Vitamin C using multi-encapsulation method. Specifically speaking, cosmetic ingredients and concentrated active ingredient like vitamin C are well blended into powder using advanced multi-encapsulation method to achieve maximum level of stabilization. The powder will convert to liquid when applied to skin so that penetration of stabilized Pure Vitamin C to the skin is easily performed and maximize its effect.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

This invention was developed to solve the problem of the instability of the previously utilized functional or active ingredients, thus to make improved functional cosmetic products. Recent world-wide trend of cosmetic development strongly suggests that our customers not only want the cosmetic products with general functions, like protecting the skin from UV light and beauty culture, but the ones with various other special functions. The vast amount of research is conducted on the incorporating those special functions into the cosmetic product. However, those active ingredients which would perform the special functions are physically, and chemically unstable towards the atmosphere, so they tend to decompose due to oxidation, and other chemical processes. This decomposition leads to discoloring and undesired odor. Because of this, previous methods could only incorporate low dosage of the active ingredients into the cosmetic products.

To overcome this disadvantage, we have developed the technique called multi-encapsulation to stabilize one of the active ingredients, pure Vitamin C by protecting it from deactivating environment. This enables to maximize the functions of pure Vitamin C as a cosmetic active ingredient.

### BRIEF SUMMARY OF THE INVENTION

With this disclosure, we report a method called multi-encapsulation to stabilize rather unstable pure Vitamin C, which is highly desired ingredient in cosmetic industry. The brief description of the process is as followed. Firstly, the active ingredient like vitamin C is stabilized with cosmetic base ingredients as a liposome. The second step is to encapsulate the above active ingredient liposome with micro-powder, and lastly the micro-powder is coated with porous powder which is encapsulated with emollient. The final product is dried by freeze-dry technique to remove all the moist, and this is the end of multi-encapsulation method to stabilize the active ingredient, which in this case is pure not derivate vitamin C.

The novel multi-encapsulation is distinct from other available methods in a way that it protects physically and chemically unstable active ingredient which is prone to undergo decomposition upon the contacts with moist or ions in the air. Thus the multi-encapsulation method effectively blocks the interaction between the active ingredients and those that may decrease the activity of the ingredient, and greatly prolong the life span of the active ingredients. The encapsulation is removed upon physical contact so that the active ingredient is dissolved in solution phase and transferred to the skin.

### BRIEF DESCRIPTION OF THE SEVERAL VIEWS OF THE DRAWING

Not Applicable

### DETAILED DESCRIPTION OF THE INVENTION

The main purpose of this invention is to develop a new method to protect the unstable active ingredient, pure Vitamin C, by encapsulation to produce the stabilized powder. This, blended with base formulation, would be dissolved and transferred to skin more effectively, maximizing the functional benefits of pure Vitamin C. Furthermore, the stabilized pure Vitamin C powder can be incorporated with high dosage to increase its activity.

The cosmetic products have attracted the attention of women of all nations not only due to their properties like protecting the skin from external factors like dryness, sun-light, etc, and preserving the purity of the skins, and keeping the skin moisture, preventing from being rough, but also their colors and fragrances, providing the women with more beauty.

However, the desire of the customers keeps evolving, hoping that the more special functions will be provided in the cosmetic products. Therefore, the cosmetic industry started to focus their research on how to incorporate the active ingredients into their products. Albumin, lecithin, licorice, Vitamin C, Vitamin C derivatives, retinol, tocopherol, salicylic acid, benzoyl peroxide, azelaic acid are the several examples of the active ingredients that the cosmetic industries are interested in, and the most well-known methods for protecting the active ingredients is to make them as liposome. According to the reference, C&T, 105, May, 1990, p65-78, C&T, 109, 1996, Fragrance/J, 1984, liposome method can temporally protect the active ingredients from the deactivating environment, so the active ingredients can have prolonged life time. Also the liposome is a good skin-penetrating agent, further increasing the efficiency of the active ingredients transfer. However, this liposome method alone could not stabilize all the active ingredients.

Here, we disclose the new method to protect unstable pure Vitamin C. Previously, there are a few methods reported by other research groups, which include “a lotion that contains pure Vitamin C dissolved in excess alcohols”, “a lotion that pure Vitamin C and some special brownish ingredient extracted from a plant, mixed together”, and “a cream that contains pure Vitamin C in water in silicone type”. All these previous methods utilize airless tube or pipette vessels to block the contacts of pure Vitamin C from the external deactivating agents. Although these methods seemed pretty successful as the protecting agents, they showed the decrease in the protection towards high temperature and the exposure to air. Also there is a method that comes with a week worth of powdered pure Vitamin C, and the cosmetic water separately, so that one will mix them together just before use. This method showed improved protection compared to other methods. Nevertheless, this method lacks convenience, and it is hard to implant a high dosage of pure Vitamin C. Even when the high dosage is incorporated, the effective activity of pure Vitamin C is lowered due to the fact that pure Vitamin C tends to undergo decomposition or oxidation by heat, light, moist and air, leading to discoloring and precipitation.

To come up with better solution, we invented a stabilization method called multi-encapsulation technique to wrap around the unstable pure Vitamin C with protecting capsule and blended with base formulation. This stabilized pure Vitamin C is kept as powder and when applied, the pure Vitamin C is dissolved into skin safely. This is a highly improved technique compared to the precedent methods, since the pure Vitamin C can be applied with high concentration so that its activity such as anti-wrinkling and whitening effects can be maximized.

To stabilize pure Vitamin C so that it can act as a powdered active ingredient, we used the multi-encapsulated method. As the first step, we must make the pure Vitamin C as a liposome. In doing so, we had to carefully dissolve it and make the liposome since we used concentrated pure Vitamin C. Secondly we encapsulate this liposome with micro-powder. Independently, we prepared the emollient encapsulated with porous powder, and coated it with micro-powder prepared in second step. Lastly, we removed all the moist by drying the multi-encapsulated powder containing pure Vitamin C that gives a special function to cosmetic product. We have found that multi-encapsulated powder with high moisture content display less stability, so it seems drying procedure is one of the crucial steps in this method.

To test the stability of the product, we conducted the following experiments

i) The Stability Towards Physical Contact:

We used J. Engelsmann's volumeter to test the stability of the product towards physical shock. This device is frequently used to test the stability of the powder. The device gives impulse to the powder by vibrating the powder up and down, and measuring the degree of coagulation as a result of the impulse tells about the degree of stabilization.

Followings are the detailed procedure:

40 grams of the powder was added to a 100 ml flask, and vibrated 100 times.

The above powder was filtered with filter funnel size 50 (300 um).

The mass of the substance left on the filter which is the destroyed capsules was measure and the weight percentage was calculated.

ii) The Moistening Effect:

We selected a group of people who would testify the moistening effect of the invented products.

iii) The Life Time:

To examine the heat stability of the powder, we measured the amount left after six months at 25° C. and three months at 40° C. using HPLC

iv) The Increase in Stability by Drying:

To improve the stability, we used Freeze-dry method to get rid of the moist.

This disclosure demonstrates the multi-encapsulation method to protect unstable active ingredients like pure Vitamin C from heat, light, moisture and air. The merit of this method is that it enables the concentrated active ingredient to be used in cosmetic product due to its stability towards heat and physical shock.

The invented powder is consisted of 1.0˜30 percent by weight, porous powder, 0.1˜20% by weight, liposome, 7.1˜15% by weight, silica silate, 60˜90% by weight, solvent, and 0.1˜15% by weight, the active ingredient.

The porous powder used in this procedure is polymethyl-methacrylate and silica. We used 1.0˜30% by weight of this porous powder because using less than 1.0% by weight results in decrease in the degree of encapsulation of liposome leading to the loss of stability. On the other hand, using more than 30% by weight gives uncomfortable dryness due to the strong adsorption power of porous powder. We are using Sunsil-130, Silica Microbead L-15000, Microsphere BPA-500, or Micropearl M-101 for the source of polymethyl-methacrylate and silica.

For the liposome component which we used 0.1˜20% by weight, using more than 20% by weight resulted in the difficulty in the step where the liposome was encapsulated by the porous powder. The liposome helps to stabilize the active ingredient in solution phase and provides smoothness with the help of emollient when applied to skin. Also it has the function as to prolong the moistening power into the cosmetic product by controlled release. The main component of the liposome is squallene, glycerol trioctanoate, capril caprilic triglyceride, cyclomethicone, jojoba oil, tocopherol acetate, lecithin, polyglyceryl myristate, glycerin, stearic acid, cetanol, phytosphigosine, polyglyceril methacrylate, sodium hyaluronate, distilled water, etc were mixed together. This liposome provides initial protection.

For the base formulation, we used silica silate as much as 7.1˜15% by weight. Using less than 7.1% by weight results in unstable solution phase, and increase the chance of coagulation by physical shock. On the other hand, using more than 15% by weight makes too concentrated solution that it give dry feeling and leave nebula on the skin. For the source of silica silate, we used commercially available Cabosil TS-530, Erosil R-972 and others that have surface area of larger than 100 m²/g.

For the solvents, we used water and one or the mixture of two from the following commercially available chemicals like glycerin, diglycerin, triglycerin, propyl glycerin, 1,3-butyl glycol, hexylene glycol, sorbitol, pentenol, sodium hyaluronate, trehalose. We used 60˜90% by weight of the solvents listed above because using less than 60% by weight results in solubility issue, and using more than 90% by weight gives less stable powder.

For the active ingredient, we could use any of pure Vitamin C, albumin, lecithin, glycyrrhizin acid, retinol, retinol palmitate, vitamin E, vitamin E acetate, salicylic acid, benzoyl peroxide, and azelaic acid, We used 0.1˜15% by weight. The precise amounts of those active ingredients were determined by HPLC.

The detailed procedure of making our encapsulation product is as follows.

1. The active ingredient and the solvent was made as liposome. This was mixed homogenously with silica silate using an ultra-fast mixer.

2. To the porous powder, emollient was added little by little, and again mixed homogenously making them powder using the ultra-fast mixer.

3. The above products were again mixed homogenously making them powder using the ultra-fast mixer, and the water was removed by the Freeze-dry technique.

The below shows the detailed experiments, and tests.

Several samples with different chemical composition were synthesized by ultra-fast mixer as shown in Table 1. The total of 7 samples were prepared. The first three whose composition ratio of the chemicals were outside the required ratio, were labeled as Comparison 1, 2, and 3, and the last four with correct compositions were labeled as Good sample 1, 2, 3, and 4. Then we have tested these samples on their fineness, moisturizing, and their degree of purity as a function of time. The followings are the procedures and the grades for those samples prepared.

The procedure for the tests, and their grades.

I) The fineness: 20 grams of the samples were filtered through the filter funnel with size 50 (300 um), and the filterate was weighed again.

A: 95% or more passed through the filter

B: 90 to 95% passed through the filter

C: 85 to 90% passed through the filter

D: 80 to 85% passed through the filter

E: 75 to 80% passed through the filter

F: 75% or less passed through the filter

II) Moisturizing activity: The samples were prepared and applied to the skin and tested how moisture the samples were

A: Very moisture

B: Moisture

C: Average

D: Dry

E: Very Dry

III) The degree of purity over the time: To determine the degree of purity over the time, the samples were left at 25° C. for six months and then injected to HPLC to measure how much pure samples were left after 6 months.

A: The purity of 95% or higher

B: The purity of 90˜95%

C: The purity of 85˜90%

D: The purity of 80˜85%

E: The purity of 75˜80%

F: The purity of 75% or below

IV) The stability towards the physical shock: We used J. Angelsman(?)'s scamvolumemeter to test the stability of the product towards physical shock. This device is frequently used to test the stability of the powder. The device gives impulse to the powder by vibrating the powder up and down and the degree of stabilization is measured by the degree of coagulation as a result of the shock. Followings are the detailed procedure;

*40 grams of the samples were placed in 100 ml flask, and vibrated 100 times

The above powder was filtered with filter funnel size 50 (300 um).

The mass of the substances left on the filter which is the destroyed capsules were measured and the weight percentage was calculated.

A: 5% or less destroyed

B: 5˜10% destroyed

C: 10˜15% destroyed

D: 15˜20% destroyed

E: 20% or more destroyed

Table 3. The Results of the Tests

The outcome of the testing 7 samples showed good stability of the encapsulated pure Vitamin C against physical shock, over the time, and high degree of fineness and good moisturizing activity.

| | |
|---|---|
| Comparison | Good Samples |

| Chemicals | | 1 | 2 | 3 | 1 | 2 | 3 | 4 |
|---|---|---|---|---|---|---|---|---|
| Silica Silate | | 3.0 | 5.0 | 20.0 | 7.1 | 10 | 15 | 8.5 |
| Glycerin | | 20 | 20 | 20 | 40 | 50 | 20 | 58.7 |
| 1,3-butylenes glycol | | 15 | 15 | 15 | 5 | 10 | 15 | 5 |
| Pentenol | | 0.5 | 0.5 | 0.5 | 0.5 | 0.5 | 0.5 | 0.5 |
| Paraben | | 0.3 | 0.3 | 0.3 | 0.3 | 0.3 | 0.3 | 0.3 |
| Distilled Water | | The | The | the rest | Rest | Rest | rest | — |
| | | rest | rest | | | | | |
| Liposome | | 0.05 | 25 | — | 0.3 | 4.0 | 8 | 5 |
| Porous Powder | | 0.1 | 0.5 | 0.8 | 1.0 | 15 | 20 | 12 |
| Pure Vitamin C | | 0.01 | 0.05 | 20 | 5 | 10 | 15 | 10 |
| Grading | Fineness | E | D | A | A | A | A | A |
| | Physical shock | E | D | A | A | A | A | A |
| | Moisture | D | A | E | B | B | C | A |
| | Purity over time | C | C | C | C | B | B | A |

The Detailed Procedure:

1. The active ingredient, liposome, silica silate and the solvent mixed homogenously using an ultra-fast mixer.

2. To the porous powder, emollient was added little by little, and again mixed homogenously making them powder using the ultra-fast mixer.

3. The above products in step 2 and 3 were again mixed homogenously making them powder using the ultra-fast mixer.

4. Water was removed by the Freeze-dry technique.

Those three Comparison samples that had the incorrect chemical composition were graded below average for the fineness of the powder and the purity over the time, meaning the stability of encapsulated pure Vitamin C was low. However, four Good samples showed improvements for all of the categories of the tests, demonstrating the power of the multi-encapsulation technology.

The followings are the conclusion drawn from the testing. The idea to block the interaction between the water and the active ingredient, pure Vitamin C by covering it by liposome capsules powder, indeed worked well, and also removing water which resided in the capsules by Freeze-dry technique, prolonged the life-time of the pure Vitamin C.

Based on the results of the testing, we developed following four products.

1. Powder with Whitening Acitivity.

| | |
|---|---|
| Silica Silate | 8.0% |
| Glycerin | 60.0% |
| Distilled water | the rest |
| 1,3-butylene glycol | 5.0% |
| paraben | 0.3% |
| pentenol | 0.5% |
| liposome | 6.0% |
| porous powder | 10.0% |
| Pure Vitamin C | 10.0% |

The above product contains 10% of pure Vitamin C which has superior whitening effect. Since the pure Vitamin C is stabilized as the capsules, the life time of the pure Vitamin C is very long. Even when heated at 40° C. for 3 months, the purity of the active ingredient was higher than 90% (determined by HPLC)

2. Anti-Wrinkle Powder

| | |
|---|---|
| Silica Silate | 10.0% |
| Glycerin | 63.0% |
| Distilled water | the rest |
| 1,3-butylene glycol | 5.0% |
| paraben | 0.3% |
| pentenol | 1.0% |
| liposome | 8.0% |
| porous powder | 12.0% |
| Pure Retinol (1,420,000 IU/g) | 0.2% |

The above product contains Retinol (3,000 IU) which has anti-wrinkle activity.

3. Acne Powder

| | |
|---|---|
| Silica Silate | 10.0% |
| Glycerin | 20.0% |
| Distilled water | the rest |
| 1,3-butylene glycol | 10.0% |
| paraben | 0.3% |
| pentenol | 0.1% |
| liposome | 8.0% |
| porous powder | 12.0% |
| Salicylic Acid | 0.5% |

The above product contains salicylic acid which prevents acne.

4. Keratin-Removing Powder

| | |
|---|---|
| Silica Silate | 8.5% |
| Glycerin | 40.0% |
| Distilled water | the rest |
| paraben | 0.3% |
| pentenol | 0.5% |
| liposome | 8.0% |
| porous powder | 12.0% |
| AHA | 0.5% |

The above product contains high concentration of AHA which can remove keratin. Since AHA is stabilized in the capsules, the life time of AHA is very long. Even when heated at 40° C. for 3 months, the purity of the active ingredient was higher than 90% (determined by HPLC).

As stated above, the new technology to encapsulate the active ingredients to stabilize them from decomposition and keep them as fine powder was developed. The cosmetic product were made from this stabilized active ingredients blended with the base formulation. These active ingredients in the capsules were not destroyed by physical shocks and penetrated well into skin, resulting maximum anti-wrinkle, whitening, acne, and keratin-removing effect. Not only has this method provided the stability of the active ingredients, but also the ease for use, and excellent moistening effect.

We claim:

1. The composition of the base formulation for encapsulated pure Vitamin C is porous powder 10˜30% by weight, liposome 0.1˜20%, silica silate 7.1˜15%, solvent 60˜90%, pure Vitamin C 0.1˜15%.

2. Polymethyl-metharylate and silica are mixed and used as the porous powder mentioned in claim#1.

3. The liposome mentioned in claim#1 is consisted of mixture of squallene, glycerol trioctanoate, capril caprilic triglyceride, cyclomethicone, jojoba oil, tocopherol acetate, lecithin, polyglyceryl

4. The following solvents in claim#1 are used: glycerin, diglycerin, triglycerin, propyl glycerin, 1,3-butyl glycol, hexylene glycol, sorbitol, pentenol, sodium hyaluronate, trehalose, and distilled water.

5. Other active ingredients are pure Vitamin C, albumin, lecithin, glycyrrhizin acid, retinol, retinol palmitate, tocopherol, tocopherol acetate,salicylic acid, benzoyl peroxide, and azelaic acid.
